(19) United States
(12) Patent Application Publication
(10) Pub. No.: US 2013/0150767 A1
(43) Pub. Date: Jun. 13, 2013

(54) VASCULAR ACCESS DEVICE FOR HEMODIALYSIS

(71) Applicants: Eduard Tsyrulnykov, Mequon, WI (US); Erin Lipkowitz, Mequon, WI (US)

(72) Inventors: Eduard Tsyrulnykov, Mequon, WI (US); Erin Lipkowitz, Mequon, WI (US)

(21) Appl. No.: 13/525,393

(22) Filed: Jun. 18, 2012

Related U.S. Application Data
(60) Provisional application No. 61/499,619, filed on Jun. 21, 2011.

(51) Int. Cl. A61M 1/14

(52) U.S. Cl. CPC A61M 1/14
USPC 604/5.04

(57) ABSTRACT

Single needle vascular access systems, devices and methods for use in hemodialysis and apheresis procedures. A single needle vascular access device includes a body having an upper and lower passage with both in fluid communication with a vascular dilator. A cannulation needle is guided through the lower passage and vascular dilator to cannulate a graft or fistula. Following cannulation, the dilator is gently introduced into the vessel. The cannulation needle is removed, and a venous tube is introduced through the upper passageway through the vascular dilator and into the fistula or graft. Blood is then removed from the body through the vascular dilator side orifices and returned through the venous line. Depending on the clinical situation, the vascular dilator can be used with one or both needles of a conventional two needle system. In the later case, the vascular dilator may be connected to the dialysis tubes using conventional standard connectors.

### BACKGROUND

1. Technical Field

The invention relates to methods and devices to facilitate bi-directional blood flow through a single access point. More particularly the invention relates to extracorporeal hemodialysis of a patient's blood with a single cannulation.

2. Description of the Related Art

Historically, kidney diseases have been of critical concern to human life. Many kinds of kidney diseases interfere with the function of the kidney such that the kidney ceases to remove waste and excess water from the blood. When the kidney is sufficiently impaired that large portions of the waste products and water are not removed from the blood, the life of the patient cannot be preserved unless a way is provided for artificially performing the function of the impaired kidney. Even today, the same general procedure is used for dialyzing patients' blood that was used very early in the treatment of kidney disease.

For example, the most commonly accepted practice for dialyzing a patient's blood extracorporeally requires the surgical creation of a vascular access such as arterio-venous fistula (artificial connection between artery and vein) or graft. Grafts are much like fistulas in most respects, except that an artificial vessel is used to join the artery and vein. The graft usually is made of a synthetic material, often PTFE, but sometimes chemically treated, sterilized veins from animals are used. Grafts are inserted when the patient's native vasculature does not permit a fistula.

Sufficient blood flow for dialysis is then obtainable by cannulation of fistula or graft with large bore needles. Normally, two hollow needles are used to perform two cannulations on the patient hemodialysis access (fistula or graft) so that two point blood-communication sites exist simultaneously in the patient. Conventionally, blood is withdrawn from one part of the punctured hemodialysis access, forced through a hemodialyzer and thereafter forced into the remaining part of the hemodialysis access. The needles have to be substantially distant from one another to prevent recirculation of blood between the access sites.

It is well-known that the life expectation and effective function of a hemodialysis access is inversely related to the number of cannulations. Tissue repeatedly subjected to the trauma of cannulation is much more susceptible to inflamation neointimal hyperplasia, thrombosis and pseudo aneurism development. Such trauma may result in destruction of the graft or fistula triggering a requirement for another expensive operating procedure to replace the graft or fistula so that routine treatment may be resumed.

Systems for hemodialysis access to a fistula or graft cannulation can generally be divided on the following groups.

Two Needle Cannulation

Two needle cannulation procedures are well known being the first developed and the most widely used system at the present time. They are able to provide flow rates above 600 ml /minute to minimize patient clinical procedure time. The disadvantages of these systems compared to a single cannulation approach, include pain and trauma to access, the risk for developing complications including pseudoaneursm, post treatment bleeding, and outflow stenosis. An example of this approach can be found at: http://www.baxter.com/healthcare_professionals/products/hemodialysis_fistula_needles.html

Sequential Flow Single Needle Cannulation

A sequential flow single needle cannulation system uses a single needle cannulation for both removing and replacing blood by alternating between forward and reverse flow through the single needle. The benefits of this system are that the one needle cannulation decreases trauma and pain in the area of the cannulation since only one cannulation is required. A disadvantage is that the high recirculation rate results in extensive remixing of treated and untreated blood, which reduces the effectiveness of the dialysis procedure. An example of this approach is disclosed in U.S. Pat. No. 4,940,455.

Telescoping One Needle Cannulation

Another single needle system includes a one needle cannulation uses dual coaxial tubes to form an integral 2 lumen needle to simultaneously add and remove a patient's blood with one introduction point. The benefit of this system is that only one needle cannulation is required thereby decreasing the trauma/pain in the area of cannulation. The disadvantages of this procedure include that using the cannulation needle as an outer tube (without using a dilator) which restricts blood flow through the system to an average flow of 300-400 ml/minute. These low flow rates are not acceptable for the most hemodialysis patients. Furthermore, using a rigid inner tube material makes the device inappropriate to use in difficult geometry access such as stenotic, snail, torturous and angled fistula. An example of this system is described in U.S. Pat. No. 4,037,599.

### SUMMARY OF THE INVENTION

Devices, systems and methods are provided for a single needle vascular access for routine use in hemodialysis and apheresis procedures using a single access point. The vascular access device includes a body having an upper and lower passageway in fluid communication with a vascular dilator. The lower passageway is substantially straight linear through the body to allow insertion and withdrawal of the cannulation needle. An end of the lower passageway is provided with a small bore Tuohy Adapter. The upper passageway intersects the lower passageway as a side branch, forming a Y or V shape within the body. The angle between the upper and lower passageway is preferably about 15 degrees. The upper passageway is sized to allow insertion of the venous tube for replacing processed blood to the patient. An upper passageway end is provided with a large bore Tuohy Adapter to secure the venous tube.

A vascular dilator is removably fastened to the body and is located at or near the intersection of the upper passageway with the lower passage. The vascular dilator is size to sequentially receive the cannulation needle and the venous tube. The vascular dilator distal end has an open tip to allow the cannulation needle to pass through and access the fistula or graft. The front opening is preferably sized to allow the cannulation needle to pass freely without any excess space to allow solid material to collect. The vascular dilator tip is beveled, blunt or rounded to stretch the opening created by the cannulation needle, without cutting or tearing the tissue. The vascular dilator also has side orifices to allow blood to enter the interior of the vascular dilator. The diameter of the vascular dilator may be varied depending upon the blood flow rate desired for the procedure.

A cannulation needle is guided through the lower passageway and vascular dilator to cannulate a graft or fistula. A side screw is used to prevent undesired movement of the cannulation needle. Following cannulation, the dilator is gently introduced into the fistula or graft. The cannulation needle is removed. A venous tube has a narrow end with end and side openings. The venous tube narrow end is introduced through the upper passageway through the vascular dilator with non-linear movements and into the fistula or graft. The relatively small angle less than 30 degrees and preferably approximately 15 degrees between the upper and lower chamber helps minimize the pressure drop and eliminate tube constriction at the bend. The venous tube narrow end is then advanced approximately 10 mm beyond the vascular dilator tip. As the venous tube is freely rotatable within the body and vascular dilator, the venous tube may be adjusted to the geometry of the particular access. Upon securing the venous tube with the large bore Tuohy Adapter, blood is then removed from the patient through vascular dilator side orifices into the lower passageway and sent through the arterial line connected to the lower passageway for transfer to the hemodialysis machine. The patient's blood is then returned through the venous tube and discharged through the side and end orifices of the venous tube into the patient's blood stream.

### OBJECTS OF THE INVENTION

It is a primary object of the present invention to provide an improved method of extracorporeal hemodialysis using a single cannulation for each treatment.

It is another object of the present invention to provide a single entry needle to reduced damage to a graft or fistula.

It is yet a further object of the present invention to provide a single access dialysis needle having two lumens and a volume capacity at or above 600-700 ml/min for blood removal.

It is yet a further object of the present invention to provide a procedure to reduce patient time in clinical treatment.

It is yet a further object of the present invention to provide a small diameter cannulation needle to reduce patient pain

It is yet a further object of the present invention to provide a small diameter cannulation needle to reduce damage to graft or fistula

It is yet a further object of the present invention to provide a rotatable venous tube to assist threading into a vessel with having a tortuous path such as a torturous fistula, snail fistula or difficult geometry fistula.

It is yet a further object of the present invention to provide a ring or marker band locatable by standard instrumentation at or near the venous tube tip.

It is yet a further object of the present invention to provide larger diameter dilator tubes to be used as a part of a device or simultaneously in combination with standard dialysis needles and connecting for two needle systems.

It is yet a further object of the present invention to provide larger diameter venous tubes.

It is yet a further object of the present invention to provide a high blood flow rate.

It is yet a further object of the present invention to minimize pressure in venous segment of the system, which is contributing factor in developing of stenosis of a hemodialysis access.

It is yet a further object of the present invention to provide a vascular dilator that will distend tissue of graft or fistula after cannulation without causing additional trauma, bleeding or pain.

These and other objects and features of the present invention will become more fully apparent from the description below.

### BRIEF DESCRIPTION OF THE DRAWINGS

FIG. 1 provides a perspective view of the device in place on a patients arm during dialysis.

FIG. 2A provides a perspective view of the body with its associated parts.

FIG. 2B provides another perspective view of the body.

FIG. 3A provides a side view of the body with the cannulation needle installed.

FIG. 3B provides a detail of a cross-sectional view of the body showing the securing screw.

FIG. 4 provides a side view with the venous tube in place.

FIG. 5 provides a cross-section of the device inside the hemodialysis access with venous tube installed.

FIG. 6 provides a cross-section of the vascular dilator with the cannulaton needle installed.

FIG. 7A provides a view of the vascular dilator with side orifices on cannula wall.

FIG. 7B provides a view of an alternate embodiment of the vascular dilator.

FIG. 7C provides a view of the cannulation needle with the blood flow detector.

FIG. 7D provides a perspective view of the venous tube.

FIG. 7E provides a detail view of the vascular dilator with a beveled end.

FIG. 7F provides a detail view of the vascular dilator end with a rounded end using the annular space as a conduit for blood withdrawal.

FIG. 7G provides a detail view of the venous tube end with side orifices.

FIG. 8 provides a perspective view of blood flow detector.

### DETAILED DESCRIPTION OF EXEMPLARY EMBODIMENTS

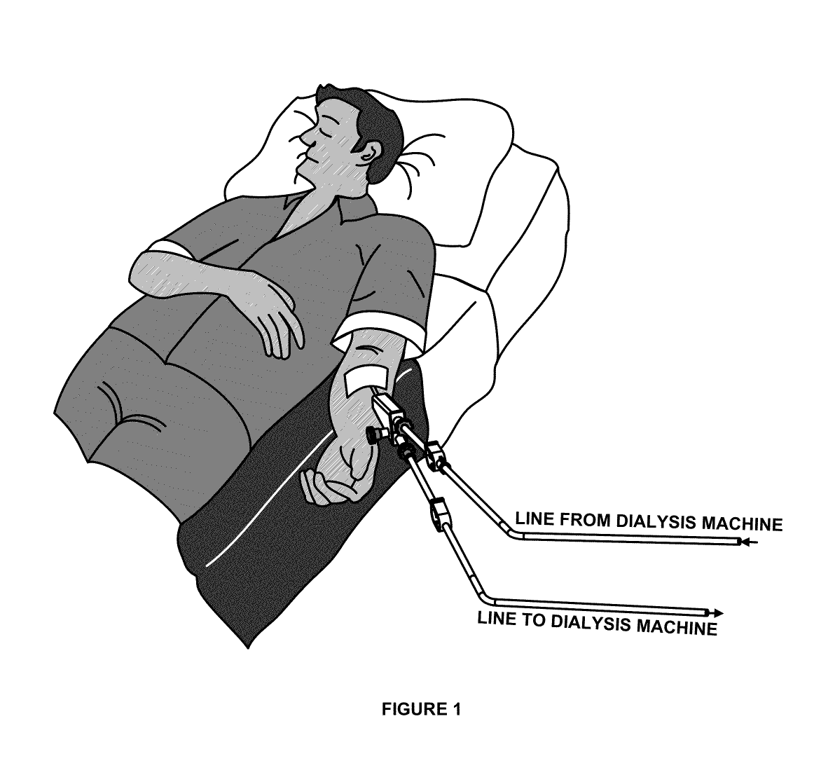

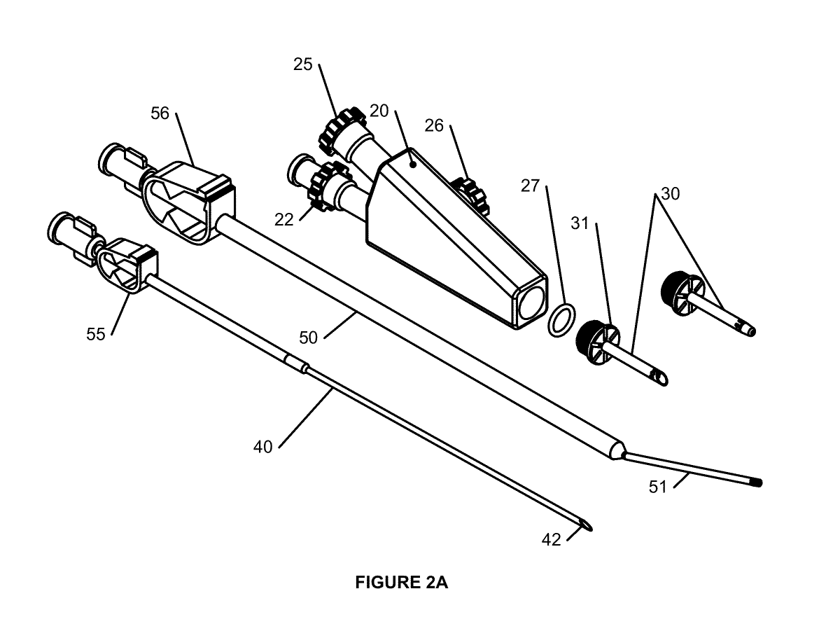

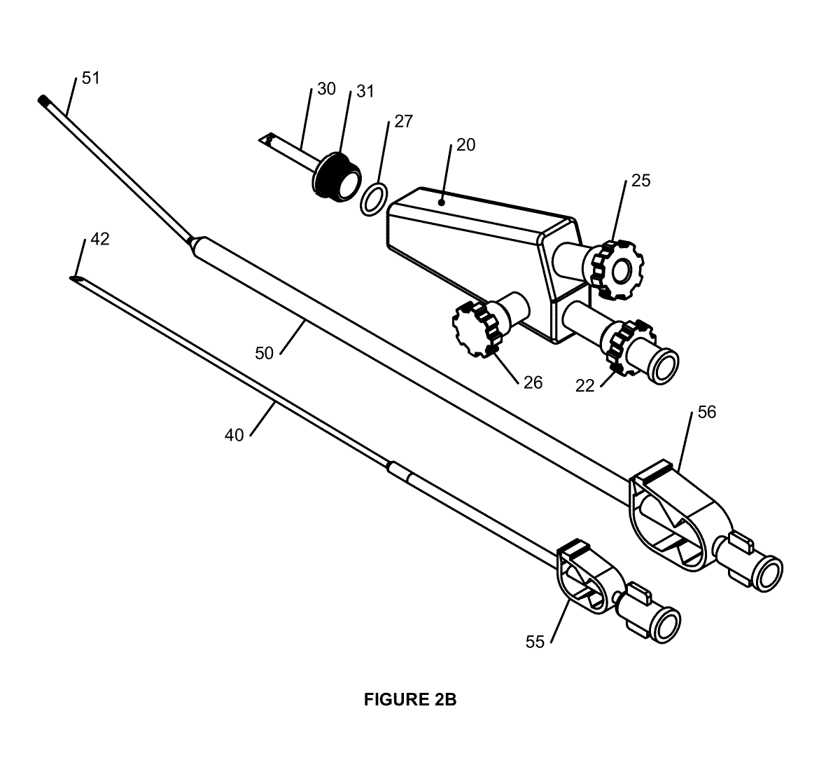

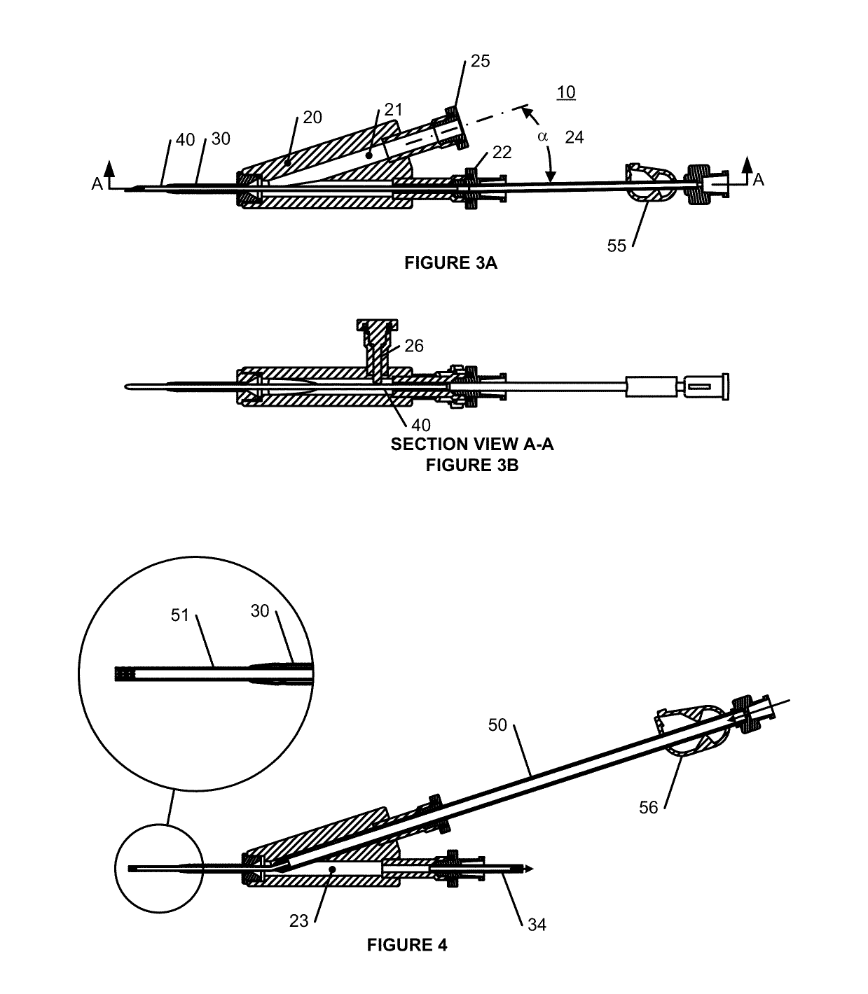

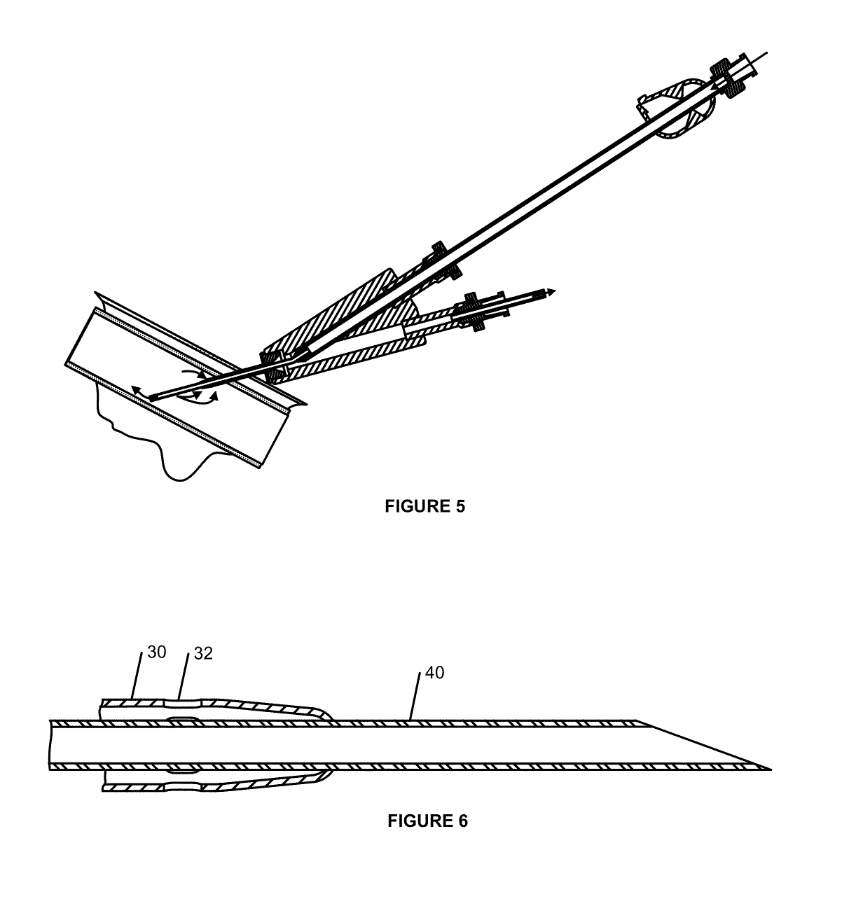

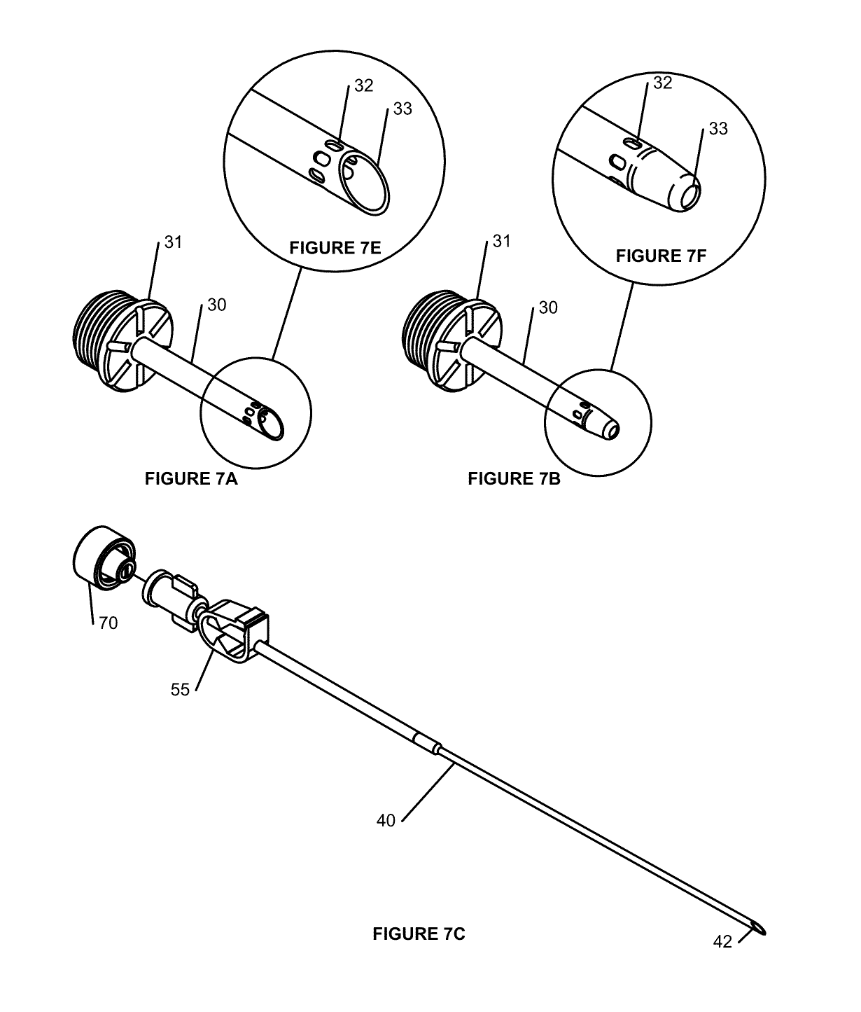

The vascular access device 10 is shown situated on a patient's arm in FIGS. 1-4. The vascular access device 10 includes a body 20 having an upper passageway 21 and lower passageway 23 in fluid communication with a vascular dilator 30. The lower passageway 23 is substantially straight linear through the body 20 to allow insertion and withdrawal of the cannulation needle 40. An end of the lower passageway 23 is provided with a small bore Tuohy Borst adapter 22. The small bore Tuohy Borst adapter 22 is provided with an adapter such as a Luer taper fitting to connect to a arterial tube 34 to carry blood to the dialysis machine. The upper passageway 21 intersects the lower passageway 23 as a side branch, forming a Y or V shape within the body 10. The angle alpha 24 between the upper and lower passageway is generally less than 30 degrees and preferably about 15 degrees. The small angles minimize pressure drop at the intersection of the passageways and makes venous tube 50 easier to position. The upper passageway 21 is sized to allow insertion of the venous tube 50 for replacing processed blood to the patient. An upper passageway proximal end is provided with a large bore Tuohy Borst adapter 25 to secure the venous tube 50. The term Tuohy Borst adapter is used as a well known descriptor of an adapter to provide a seal between an instrument or tubing and the body 20. Any adapter providing substantially the same functionality may be used.

A vascular dilator 30 proximal end is removably fastened to the body and is located at or near the intersection of the upper passageway 21 with the lower passageway 23. The vascular dilator 30 may be within a range of 8 to 16 french catheter size attached to a muzzle 31 at or substantially near the intersection of the upper and lower passageways. The muzzle 31 is threaded to mate with corresponding threads on the body 20 front face. An O-ring 27 is provided between the muzzle 31 and the body 20 to provide a tight seal. The vascular dilator 30 is sized to sequentially receive the cannulation needle 40 and the venous tube 50. The vascular dilator 30 has an open tip to allow the cannulation needle 40 to pass freely without any excess space to allow solid material to collect. The vascular dilator tip 33 may be blunt or rounded to stretch the opening created by the cannulation needle 40, without cutting or tearing the tissue. The vascular dilator tip 33 may be beveled or rounded which could be used in a standard configuration or in a buttonhole technique cannulation in either configuration. The vascular dilator tip 33 may be tapered to a blunt or rounded end to stretch the opening created by the cannulation needle 40, without cutting or tearing the tissue.

The vascular tip 33 is beveled which could be used in buttonhole technique cannulation through and access the fistula or graft. The front opening is preferably sized to allow the cannulation needle 40 to pass freely without any excess space to allow solid material to collect. The vascular dilator tip 33 is blunt or rounded to stretch the opening created by the cannulation needle 40, without cutting or tearing the tissue. The vascular dilator 30 also has side orifices 32 to allow blood to enter the interior of the vascular dilator 30. The diameter of the vascular dilator 30 may be varied depending upon the blood flow rate desired for the procedure.

A cannulation needle 40 is guided through the lower passage and vascular dilator 30 to cannulate a graft or fistula. The small bore Tuohy Borst adapter 22 can then be tightened around the cannulation needle 40 to prevent blood from exiting the lower passageway 23. Additionally, tightening the small bore Tuohy Borst adapter 22 as well as tightening the securing screw 26 are used to prevent undesired movement of the cannulation needle 40 during cannulation. The securing screw 26 is tightened to hold the cannulation needle 40 in place during dilation, and then released for withdrawal of the cannulation needle 40. Following cannulation, the vascular dilator 30 is gently introduced into the fistula or graft. The cannulation needle 40 is removed. A venous tube 50 has a narrow segment 51 with end and side openings 53. The venous tube narrow segment 51 is introduced through the upper passageway 21 through the vascular dilator 30 and into the fistula or graft. The relatively small angle less than 30 degrees and preferably approximately 15 degrees between the upper and lower passageways helps minimize the pressure drop and eliminate tube constriction at the bend. The venous tube narrow segment 51 is then advanced approximately 10 mm beyond the vascular dilator tip 33. As the venous tube 50 is freely rotatable within the body 10 and vascular dilator 30, the venous tube 50 may be turned to get through tortuous paths. Upon securing the venous tube with the large bore Tuohy Borst Adapter 25, blood is then removed from the patient through vascular dilator side orifices 32 into the lower passageway 23 and sent through the arterial tube 34 connected to the lower passageway 23 for transfer to the hemodialysis machine. To achieve single access bi-directional flow, the patient's blood supply is then returned through the venous tube 50 and discharged through the side and end orifices into the patient's blood stream. Standard tubing clamps 55 may be used to prevent or limit blood flow when desired.

In its simplest embodiment, the vascular access device 10 can be used as a standalone procedure. In this case, the dialysis fistula or graft will be cannulated by a standard 15 gauge fistula needle. The vascular dilator 30 will be introduced over the standard dialysis needle, the needle will be removed and the vascular dilator 30 will be directly connected to the dialysis lines using standard connectors.

Additional features of specific embodiments are described in greater detail below.

Body

As shown in FIGS. 1-4, the body 20 may be a substantially rectangular cube shape, alternatively the body may have any shape suitable for containing the upper and lower passageways and the appropriate angle alpha 24 between the passageways. For example, a V or Y-shaped fitting configuration would also be acceptable. The body 20 may be secured to the patient with removable Velcro strips, releasible adhesives, or attached with standard ties.

Vascular Dilator

The vascular dilator 30 size will preferably be between 8 and 16 french catheter sizes. The vascular dilator 30 has an interior conduit which alternately contains portions of the cannulation needle 40 and the narrow segment 51 of the venous tube 50, thus serving as a sheath for portions of the cannulation needle 40 and venous tube 50 within the patients tissue. The larger diameter of the vascular dilator 30 allows increased blood flow from the patient. The needle portion preferably has a muzzle 31 attached to the body 20 and a vascular dilator tip 33 with an open free end. The rim of the vascular dilator tip 33 may be beveled, blunt or rounded to prevent tissue damage when inserted into the vessel during regular cannulation. The vascular dilator tip 33 in a beveled or rounded embodiment preferably has two or more vascular dilator side orifices 32 to allow blood collection through the vascular dilator 30 during use even if the wall of the fistula or graft is sealing one of the openings. As such, it is preferable that the combined area of the vascular dilator side orifices 32 is greater than the area of the end opening of the vascular dilator tip 33. The vascular dilator tip 33 may be beveled at an acute angle to aid insertion through a tissue opening. The vascular dilator tip 33 may be either rounded or beveled at an acute angle to aid insertion through a tissue opening which will make it useful during buttonhole cannulation.

Unlike regular dialysis needles, it is safe to flip the vascular dilator 30 without risk of damaging of endothelium as there are no sharp parts. Furthermore, it is provided that as the vascular dilator 30 is inserted through the cannulation, it will distend tissue of graft or fistula without causing additional trauma, bleeding or pain. To prevent post treatment bleeding upper part of vascular dilator residing in sub epidermal layer of the skin might be covered with local hemostatic. Other medicants that may be used, as for example, a coating include medical lubricants, anesthetics, anti-microbials, disinfectants or anti-clotting agents.

Cannulation Needle

The cannulation needle 40 is preferably a standard hollow 14 or 15 gauge aspiration needle approximately 20-cm in length, with a razor sharp tip for making the cannulation. Using the smaller diameter cannulation needle helps to reduce patient pain and to reduce damage to graft or fistula as the cannulation needle tip 42 passes through tissue such as skin and the blood vessel wall. The cannulation needle 40 is substantially the same diameter as the vascular dilator tip opening to prevent tissue from entering the vascular dilator 30 during cannulation and dilation.

Venous Tube

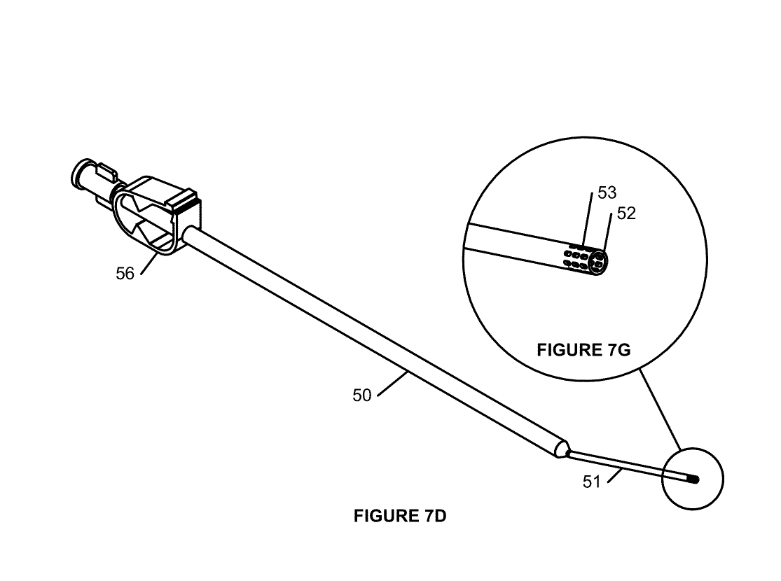

Venous tube 50 shown in FIG. 7D is composed of flexible tubing for inserting through the upper passage way 21 and into the vascular dilator 30 following removal of the cannulation needle 40. The venous tube 50 carries blood from the patient through the vascular dilator 30, body 20 and large bore Tuohy adapter 25. The venous tube 50 includes a narrow segment 51 which has sufficient rigidity for insertion into the graft or fistula. For example, the narrow segment 51 may have a 0.3 mm wall thickness. A large interior diameter narrow segment 51 is preferred. For example, in one embodiment, an internal diameter of 1.7 mm and a wall thickness of approximately 0.3 mm may be used to minimize pressure drop in venous segment of the system, which is contributing factor in developing of stenosis of hemodialysis access. A wall thickness of 0.1 mm is preferred. The venous tube tip 52, may include an strip of metal or other radio opaque material to assist identifying the location of within the graft or fistula during insertion using common medical imaging methods. The annular space between the inner surface of the vascular dilator 30 and the venous tube tip 52 forms a conduit for blood flow into the body 20 and to the arterial tube 34.

Blood Flow Detector

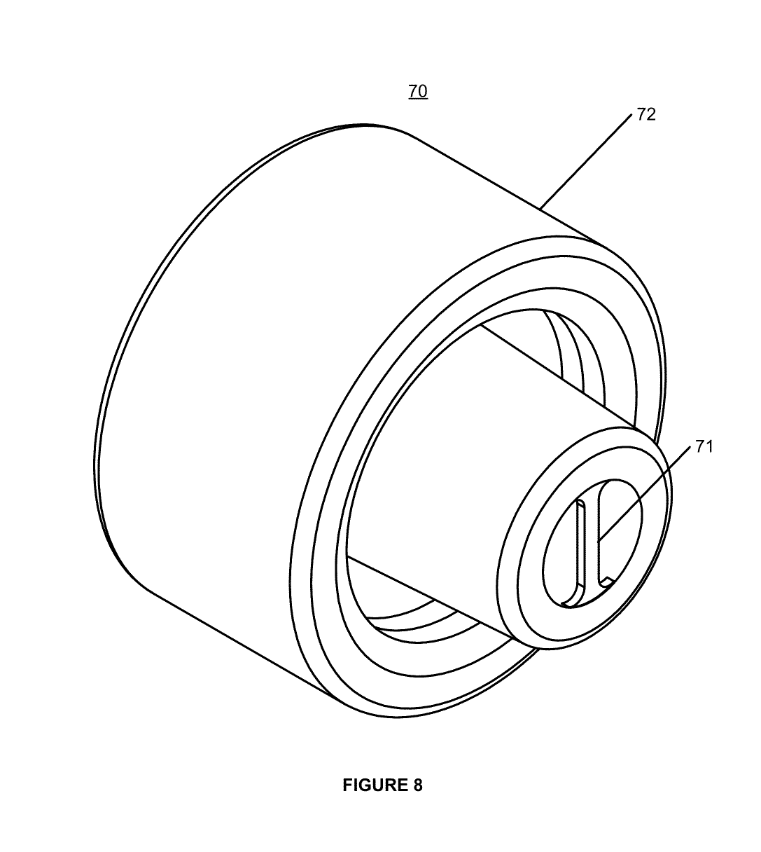

To assist medical personnel during cannulation, the vascular access device 10 may optionally include a blood flow detector 70, shown in FIG. 8, that will inform the technician when a successful cannulation has been made. The disclosed blood flow detector 70 includes a frame 72 and a vibrator 71 and relies on the patient's natural blood pressure to operate. The frame 72 containing a vibrator 71 is attached to the cannulation needle 40. The vibrator may provide a audible signal such as a whistle, if a rubber membrane is used, or a visual signal if, for example a flap valve is used. As the needle punctures a graft or fistula, blood, taking the path of least resistance, enters the cannulation needle 40. The blood displaces air in the cannulation needle, which activates the vibrator 40 providing an indication that the cannulation has been completed.

1. A method for accessing a dialysis site comprising the steps of:
a. cannulating a dialysis access site with a cannulation needle;
b. advancing a vascular dilator over the cannulation needle;
c. gently advancing the vascular dilator into the dialysis site into direct contact with the blood supply;
d. removing the cannulation needle from the dialysis site; and
e. connecting the vascular dilator to a dialysis line to provide blood to a blood dialysis unit.

2. A method to facilitate bi-directional flow during dialysis through a single access point comprising the steps of:
a. positioning a vascular access device having a body with an upper passageway and a lower passageway;
b. attaching a vascular dilator to the lower passageway at the front side of the body.
c. initiating a cannulation by temporarily passing a cannulation needle through the vascular dilator using the lower passageway within the vascular access device;
d. accessing a blood supply by cannulating a wall of a blood vessel with the cannulation needle;
e. urging the vascular dilator over the cannulation needle into contact with the blood supply;
f. threading a venous tube having a narrow segment though the upper passageway of the body;
g. advancing the narrow segment using non-linear movements through the vascular dilator into the blood vessel;
h. providing the blood supply for dialysis through the vascular dilator; and
i. returning the blood supply from dialysis through the venous tube.

3. A singular access point vascular access device to facilitate bi-directional flow comprising:
a body having an upper passageway in fluid communication with a lower passageway;
the body also having a vascular dilator attached in fluid communication with the lower passageway front side of the body;
the vascular dilator removably accepting a cannulation needle through the lower passageway;
the cannulation needle having an inner bore and a cannulation needle tip;
the upper passageway removably accepting a flexible venous tube having a narrow segment at the venous tube distal end; and
the narrow segment having a venous tube tip for extending beyond the entire length of the vascular dilator using non-linear manipulations of the venous tube.

4. The device of claim 3 wherein the vascular dilator tip includes a plurality of vascular dilator side orifices.

5. The device of claim 3 wherein the venous tube tip includes a plurality of venous tube side orifices.

6. The device of claim 3 wherein the vascular dilator tip has an end opening with vascular dilator side orifices having a total area that is greater than the area of the end opening.

7. The device of claim 6 wherein the end opening is rounded.

8. The device of claim 6 wherein the end opening is narrowed to substantially eliminate any annular space between the cannulation needle and the vascular dilator tip.

9. The device of claim 3 also having a small bore Tuohy Borst adapter attached to the lower passageway for retaining the cannulation tube.

10. The device of claim 9 wherein the small bore Tuohy Borst adapter is provided to receive an arterial tube.

11. The device of claim 3 having a securing screw at least partially within the lower passageway for securing the cannulation tube.

12. The device of claim 3 also having a large bore Tuohy Borst adapter attached to the upper passageway for securing the venous tube.

13. The device of claim 3 wherein the upper passageway and lower passageway intersect at an angle in the range of 5 degrees to 30 degrees.

14. The device of claim 3 wherein the upper passageway and lower passageway intersect at an angle of approximately 15 degrees.

15. The device of claim 3 also having a blood flow detector removably fastened to the cannulation needle to identify a successful cannulation.

16. The device of claim 3 wherein the vascular dilator includes a muzzle for removably attaching the vascular dilator to the body.

17. The device of claim 3 having an annular space within the vascular dilator in fluid communication with an arterial tube. (connects to the small bore tuohy connector)

18. The device of claim 3 also having an O-ring located between the muzzle and the body.

19. The device of claim 3 also having a metal strip on the venous tube tip for assisting with positioning.
